(19) United States
(12) Patent Application Publication
(10) Pub. No.: US 2015/0282460 A1
(43) Pub. Date: Oct. 8, 2015

(54) ANIMAL MODEL WITH HUMAN IMMUNE SYSTEM

(71) Applicant: Crown Bioscience Inc. (Taicang), Taicang (CN)

(72) Inventors: Qian Shi, Taicang (CN); Juan Zhang, Taicang (CN); Junzhuan Qiu, Taicang (CN); Xin Dong, Taicang (CN); Jiping Zha, Taicang (CN); Ziyong Sun, Taicang (CN)

(21) Appl. No.: 14/679,951

(22) Filed: Apr. 6, 2015

(30) Foreign Application Priority Data
Apr. 4, 2014 (CN) 201410135522.2

(51) Int. Cl. A01K 67/027; A61K 49/00

(52) U.S. Cl. CPC A01K 67/0271; A61K 49/0008; A01K 2207/12; A01K 2267/0331

(57) ABSTRACT

The present disclosure provides an immuno-deficient animal useful as an animal model for a human disease, wherein the animal comprises: (a) functional human immune cells; and (b) a human xenograft comprising a pathogenic human cell or tissue, wherein the human immune cells and the human xenograft meet at least one of the following criteria: i) the human xenograft expresses a threshold level of a therapeutic target for the human disease; ii) the human immune cells match with the human xenograft for at least one human leukocyte antigen (HLA) marker; and iii) the human xenograft cells expresses a desired level of the matched HLA marker. Also provides is a method of producing the animal model and use of the animal models.

### RELATED APPLICATIONS

This application relates to and claims priority benefits from CN Patent Application No. 201410135522.2, filed Apr. 4, 2014, titled “An animal disease model with human immuno-system, method of producing the animal model and the use thereof”, which is hereby incorporated by reference in its entirety.

### FIELD OF THE INVENTION

The present disclosure generally relates to an animal disease model with human immuno-system, methods of producing the animal model and methods of using such animal model.

### BACKGROUND OF THE INVENTION

Pre-clinical in-vivo analyses of a drug candidate normally carried out in animal models (e.g., using rodents) are critical for drug development. The efficacy, metabolism and toxicity data of a drug candidate obtained based on animal models can be used as guidance for clinical trials.

However, pre-clinical efficacy assessments of a human immunomodulator (e.g. human antibodies) require T cell activation in a human immune competent system, and therefore cannot be accurately studied in an animal model whose immune system differs from that of human. In the current practice, surrogate mouse immunomodulator which acts on mouse targets are tested in mouse syngenic models, to provide for simulation of effects of a human immunomodulator. However, it is often very difficult to predict whether a human immunomodulator is effective or not, due to the fact that the immune systems in human and mouse are different. When transferring the gained pre-clinical results from mouse into clinical use in humans, problems may occur, for example, that the human target may be invalid in clinical use; or that the therapeutic agent does not have the same functional properties in human as observed in mouse. It is then desirable to produce animal models that can best mimic human immune reaction, in which the effects of human immunomodulator on human disease associated cells and tissues can be studied in a more close-to-native biological context.

### BRIEF SUMMARY OF THE INVENTION

The present disclosure provides animal disease model with human immuno-system, methods of producing the animal models and methods of using such animal models.

In one aspect, the present disclosure provides animal model for a human disease, said animal model comprising an immuno-deficient animal comprising: (a) functional human immune cells; and (b) a human xenograft comprising a pathogenic human cell or tissue, wherein the human immune cells and the human xenograft meet at least one of the following criteria: i) the human xenograft expresses a threshold level of a therapeutic target for the human disease; ii) the human immune cells match with the human xenograft for at least one human leukocyte antigen (HLA) marker; and iii) the human xenograft cells expresses a desired level of the matched HLA marker. In some embodiments, the human immune cells and the human xenograft meet all three criteria.

In some embodiments, the human immune cells are derived from isolated human PBMC or activated T cells. In some embodiments, the human immune cells are derived from one human donor.

In some embodiments, the matched HLA marker is HLA-A2 and/or HLA-B. In some embodiments, the matched HLA marker is expressed in the human xenograft at a level higher than in unmatched human xenograft. In some embodiments, the human xenograft comprises a tumor cell or tumor tissue. In some embodiments, the human xenograft is derived from a human patient having the disease.

In some embodiments, the human immune cells or the human xenograft are grafted to the animal intravenously, or subcutaneously, or intraperitoneally.

In some embodiments, the animal is a mammal. In some embodiments, the mammal is a rodent, such as a mouse, a rat, a guinea pig, or a hamster. In some embodiments, the animal is mouse, a rat, a guinea pig, a hamster, a dog or a monkey. In some embodiments, the animal is mice. In some embodiments, the animal is NOD-Prkdc^{scid} IL2Rγ null mice. In some embodiments, the NOD-Prkdc^{scid} IL2Rγ null mice is selected from a group consisting of strains: NSG (NOD SCID Gamma) mice, NPG (NOD-Prkdc^{scid} Il2rg^{null}) mice, NOG mice (NOD/Shi-scid/IL-2Rγ^{null}), or NCG (NOD-Prkdc^{em26cas9d52}Il2rg^{em26cas9d22}nju) mice. In some embodiments, the animal is NPG mice. In some embodiments, the animal is NCG mice.

In another aspect, present disclosure provides a method of producing an animal model for a human disease comprising functional human immune cells and a human xenograft comprising a pathogenic human cell or tissue, comprising: a) obtaining an immuno-deficient animal; and b) grafting the animal with functional human immune cells and a human xenograft comprising a pathological human cell or tissue; wherein the human immune cells and a human xenograft meet at lease one of the following criteria: i) the human xenograft expresses a threshold level of a therapeutic target for the human disease; ii) the human immune cells match with the human xenograft for at least one human leukocyte antigen (HLA) marker; and iii) the human xenograft cells expresses a desired level of the matched HLA marker. In some embodiments, the immuno-deficient animal is depleted of active endogeneous T cells and active endogeneous B cells. In some embodiments, the immuno-deficient animal is depleted of active endogeneous T cells, active endogeneous B cells and active endogeneous Natural Killer cells. In some embodiments, the human xenograft and the human immune cells the human immune cells and the human xenograft meet all three criteria.

In some embodiments, the human immune cells are derived from isolated human PBMC or activated T cells. In some embodiments, the human immune cells are derived from one human donor.

In some embodiments, the matched HLA marker is HLA-A2 and/or HLA-B. In some embodiments, the matched HLA marker is expressed in the human xenograft at a level higher than in unmatched human xenograft. In some embodiments, the human xenograft comprises a tumor cell or tumor tissue. In some embodiments, the human xenograft is derived from a human patient having the disease.

In some embodiments, the human immune cells or the human xenograft are grafted to the animal intravenously, or subcutaneously, or intraperitoneally. In some embodiments, the human immune cells are grafted through single delivery before, after or simultaneously when the human xenograft is grafted, or are grafted through multiple deliveries before, after or simultaneously when the human xenograft is grafted.

In some embodiments, the method further comprises allowing the human xenograft to grow for a time sufficient to simulate the lesion of the human disease.

In some embodiments, the animal is a mammal. In some embodiments, the mammal is a rodent, such as a mouse, a rat, a guinea pig, or a hamster. In some embodiments, the animal is mouse, a rat, a guinea pig, a hamster, a dog or a monkey. In some embodiments, the animal is mice. In some embodiments, the animal is NOD-Prkdc^{scid} IL2Rγ null mice. In some embodiments, the NOD-Prkdc^{scid} IL2Rγ null mice is selected from a group consisting of strains: NSG (NOD SCID Gamma) mice, NPG (NOD-Prkdc^{scid} Il2rg^{null}) mice, NOG mice (NOD/Shi-scid/IL-2Rγ^{null}), or NCG (NOD-Prkdc^{em26cas9d52}Il2rg^{em26cas9d22}nju) mice. In some embodiments, the animal is NPG mice. In some embodiments, the animal is NCG mice.

In yet another aspect, present disclosure provides a method of assessing effect of an immunomodulatory agent on a human disease, comprising: a) obtaining an animal model provided herein for the human disease; b) administering the immunomodulatory agent to the animal model; and c) determining the effect of the immunomodulatory agent on the human xenograft and/or on the animal model. In some embodiments, the method further comprises detecting expression of a cytokine of interest from the human immune cell, and/or detecting the population of the human immune cells in the animal model, and/or detecting infiltration of the human immune cell to the human xenograft.

In some embodiments, the animal model for the human disease comprising an immuno-deficient animal comprising: (a) functional human immune cells; and (b) a human xenograft comprising a pathogenic human cell or tissue, wherein the human immune cells and the human xenograft meet at least one of the following criteria: i) the human xenograft expresses a threshold level of a therapeutic target for the human disease; ii) the human immune cells match with the human xenograft for at least one human leukocyte antigen (HLA) marker; and iii) the human xenograft cells expresses a desired level of the matched HLA marker. In some embodiments, the human immune cells and the human xenograft meet all three criteria.

In some embodiments, the human immune cells are derived from isolated human PBMC or activated T cells. In some embodiments, the human immune cells are derived from one human donor.

In some embodiments, the matched HLA marker is HLA-A2 and/or HLA-B. In some embodiments, the matched HLA marker is expressed in the human xenograft at a level higher than in unmatched human xenograft. In some embodiments, the human xenograft comprises a tumor cell or tumor tissue. In some embodiments, the human xenograft is derived from a human patient having the disease.

In some embodiments, the human immune cells or the human xenograft are grafted to the animal intravenously, or subcutaneously, or intraperitoneally.

In some embodiments, the animal is a mammal. In some embodiments, the mammal is a rodent, such as a mouse, a rat, a guinea pig, or a hamster. In some embodiments, the animal is mouse, a rat, a guinea pig, a hamster, a dog or a monkey. In some embodiments, the animal is mice. In some embodiments, the animal is NOD-Prkdc^{scid} IL2Rγ null mice. In some embodiments, the NOD-Prkdc^{scid} IL2Rγ null mice is selected from a group consisting of strains: NSG (NOD SCID Gamma) mice, NPG (NOD-Prkdc^{scid} Il2rg^{null}) mice, NOG mice (NOD/Shi-scid/IL-2Rγ^{null}), or NCG (NOD-Prkdc^{em26cas9d52}Il2rg^{em26cas9d22}nju) mice. In some embodiments, the animal is NPG mice. In some embodiments, the animal is NCG mice.

In yet another aspect, present disclosure provides methods of identifying an active immunomodulatory agent for a human disease, comprising: (a) obtaining the animal model provided herein for the human disease; (b) administering a test immunomodulatory agent to the animal model; and (c) determining the effect of the test agent on the human xenograft; wherein therapeutic improvement in the human xenograft indicates an active immunomodulatory agent.

In some embodiments, the animal model for the human disease comprising an immuno-deficient animal comprising: (a) functional human immune cells; and (b) a human xenograft comprising a pathogenic human cell or tissue, wherein the human immune cells and the human xenograft meet at least one of the following criteria: i) the human xenograft expresses a threshold level of a therapeutic target for the human disease; ii) the human immune cells match with the human xenograft for at least one human leukocyte antigen (HLA) marker; and iii) the human xenograft cells expresses a desired level of the matched HLA marker. In some embodiments, the human immune cells and the human xenograft meet all three criteria.

In some embodiments, the human immune cells are derived from isolated human PBMC or activated T cells. In some embodiments, the human immune cells are derived from one human donor.

In some embodiments, the matched HLA marker is HLA-A2 and/or HLA-B. In some embodiments, the matched HLA marker is expressed in the human xenograft at a level higher than in unmatched human xenograft. In some embodiments, the human xenograft comprises a tumor cell or tumor tissue. In some embodiments, the human xenograft is derived from a human patient having the disease.

In some embodiments, the human immune cells or the human xenograft are grafted to the animal intravenously, or subcutaneously, or intraperitoneally.

In some embodiments, the animal is a mammal. In some embodiments, the mammal is a rodent, such as a mouse, a rat, a guinea pig, or a hamster. In some embodiments, the animal is mouse, a rat, a guinea pig, a hamster, a dog or a monkey. In some embodiments, the animal is mice. In some embodiments, the animal is NOD-Prkdc^{scid} IL2Rγ null mice. In some embodiments, the NOD-Prkdc^{scid} IL2Rγ null mice is selected from a group consisting of strains: NSG (NOD SCID Gamma) mice, NPG (NOD-Prkdc^{scid} Il2rg^{null}) mice, NOG mice (NOD/Shi-scid/IL-2Rγ^{null}), or NCG (NOD-Prkdc^{em26cas9d52}Il2rg^{em26cas9d22}nju) mice. In some embodiments, the animal is NPG mice. In some embodiments, the animal is NCG mice.

The present disclosure provides mouse models that harbor human immune cells by engrafting the immuno-deficient mice with human PBMC (the Mixeno model), and use them for efficacy evaluation of immunomodulatory agents (eg. the humanized anti-PD-1 antibody). It is expected that these Mixeno models may become useful tools in immunotherapeutic antibody development, and may greatly increase the clinical translatability of animal studies.

### BRIEF DESCRIPTION OF FIGURES

FIG. 1: Relative mRNA expression level of PD-L1 (CD274) in 553 cells from breast, skin, lung, kidney, large intestine, prostate, pancreas, stomach, upper aerodigestive tract and urinary tract are shown in log₂-based format and calculated by the Robust Multi-array Average (RMA) algorithm. All data are obtained from XenoBase, a free web-based tool developed by CrownBio which combined the publically available profiling data (expression/mutation/SNP) of more than 1000 cell lines with CrownBio proprietary RNAseq and in vivo pharmacology data.

FIG. 2: Antitumor activity on Tumor Volume of Group 1 (mice engrafted with human PBMC and treated with isotype control-human IgG4), Group 2 (mice engrafted with human PBMC and treated with anti-PD-1 antibody), and Group 3 (mice treated with anti-PD-1 antibody without being engrafted with human PBMC) (n=4/Group 1; n=3/Group 2 and 3) in the treatment in the HCC-827 Mixeno NSCLC model. Each data point represents an average of data from 3 or 4 individual mice, error bars represent SEM.

FIG. 3: Antitumor activity on Tumor Volume of Group 1 (mice treated with isotype control-human IgG4 without being engrafted with T cells derived from human PBMC), Group 2 (mice treated with anti-PD-1 antibody without being engrafted with T cells derived from human PBMC), Group 3 (mice engrafted with T cells derived from human PBMC and treated with isotype control-human IgG4) and Group 4 (mice engrafted with T cells derived from human PBMC and treated with anti-PD-1 antibody) (n=5/group) in the treatment in the A375 Mixeno melanoma NOD/SCID mice model. Each data point represents an average of data from 5 individual mice, error bars represent SEM.

FIG. 4: Antitumor activity on Tumor Volume of Group 1 (mice treated with isotype control-human IgG4 without being engrafted with T cells derived from human PBMC), Group 2 (mice treated with anti-PD-1 antibody without being engrafted with T cells derived from human PBMC), Group 3 (mice engrafted with T cells derived from human PBMC and treated with isotype control-human IgG4) and Group 4 (mice engrafted with T cells derived from human PBMC and treated with anti-PD-1 antibody) (n=5/group) in the treatment in the A375 Mixeno melanoma NPG mice model. Each data point represents an average of data from 5 individual mice, error bars represent SEM.

FIG. 5A: FACS analysis of the effect of adoptive T Cell transfer (i.v.) and T cell reconstitution in NPG (NOD-Prkdc^{scid} Il2rg^{null}) Mice (n=6). The figure shows the CD3+T cell reconstitution in NPG Mice. Percentage of human T cells (human CD3+) in total cells obtained by FACS analysis were calculated for each sample, ie. blood samples collected at day 6 (mice #1, 2, 3, 4), day 17 (mice #3, 4, 5, 6) and day 20 (mice #5, 6); and spleen samples collected at day 6 (mice #1, 2), day 17 (mice #3, 4) and day 20 (mice #5, 6).

FIG. 5B: FACS analysis of the effect of adoptive T Cell transfer (i.v.) and T cell reconstitution in NPG (NOD-Prkdc^{scid} Il2rg^{null}) Mice (n=6). The figure shows the CD3+/CD8+ T cell reconstitution in NPG Mice. Percentage of human T cells (human CD3+) in total cells obtained by FACS analysis were calculated for each sample, ie. blood samples collected at day 6 (mice #1, 2, 3, 4), day 17 (mice #3, 4, 5, 6) and day 20 (mice #5, 6); and spleen samples collected at day 6 (mice #1, 2), day 17 (mice #3, 4) and day 20 (mice #5, 6). F.

### DETAILED DESCRIPTION OF THE INVENTION

The present disclosure provides animal models for a human disease, methods of producing the animal models and methods of using such animal models.

Human immune system is known to be different from a non-human animal immune system in many ways, and therefore an immunomodulator which is tested as effective on an animal immune system may not be equally effective on human.

In the present disclosure, animal models are provided which are grafted with both functional human immune cells and a human xenograft of a pathogenic cell or tissue. The human immune cells can at least partially reconstitute human immune system in the animal whose endogenous immune system is preferably compromised or deficient. With the reconstituted human immune system, the animal model can mimic human immune responses and provide a way to evaluate human immunomodulatory agents. The human xenograft, after being grafted to the animal, can simulate or mimic the human disease or a lesion of the disease which is treatable through immune modulation. In the animal models provided herein, the human immune cells and the human xenograft are selected to meet predetermined criteria, such that the animal model can better mimic the immunomodulation against the disease tissue in human. The animal models provided herein are particularly useful in evaluating in vivo effects of immunomodulatory agents on treating the human disease of interest through modulation of reconstituted human immune system in the animal.

Animal Model

One aspect of the present disclosure provides an animal model for a human disease comprising an immuno-deficient animal comprising (a) functional human immune cells; (b) a human xenograft comprising a pathogenic human cell or tissue, wherein the human immune cells and the human xenograft meet at least one of the following criteria: i) the human xenograft expresses a threshold level of a therapeutic target for the human disease; ii) the human immune cells match with the human xenograft for at least one human leukocyte antigen (HLA) marker; and iii) the human xenograft cells expresses a desired level of the matched HLA marker.

The term “animal” as used herein refers to all vertebrate animals except human, preferably a mammal, such as a dog, a pig, a rabbit, a monkey or a rodent (e.g., a mouse, a rat, a hamster, a guinea pig or such like). In certain embodiments, the animal is a mammal. In certain embodiments, the animal model is a rodent. In certain embodiments, the animal is a mouse, a rat, a guinea pig, a hamster, a dog, or a monkey.

The term “animal model” as used herein refers to any non-human animals directly or indirectly manipulated (e.g., genetically modified, or grafted with cells or tissue) to include one or more cells bearing altered or exogenous genetic information. In a particular aspect of this invention, the animal model is an immuno-compromised non-human animal capable of receiving and supporting a xenograft without mounting a graft-rejection immune response. An “immuno-compromised” animal can either be an immuno-deficient animal which is genetically deprived of endogenous T cells, B cells, NK cells or a combination thereof. Alternatively, an animal can be immuno-suppressed by biological or chemical means. Such biological or chemical means include, without limitation, immuno-suppression by repeated treatment with irradiation, mitomycin C, cyclosporine, anti-Asialo GM1 antibody, or other immuno-suppressive agents or treatments well known in the art.

In some of the embodiments, the animals or animal models of present disclosure are immuno-deficient. The term “immuno-deficient” is used herein to describe the animal whose endogenous immune system has been partly or completely compromised, such that it does not generate sufficient immune response to reject a foreign graft (such as a foreign cell or a tissue) and therefore is capable of accepting and supporting the foreign graft as self. In certain embodiments, the immuno-deficient animal is depleted of active endogenous T cells, active endogenous B cells and active endogenous Natural Killer cells. Examples of immuno-deficient animals include, for example: T lymphocytes deficient animals (e.g. BALB/c nude mice, C57BL nude mice, NIH nude mice, nude rat, etc.); B lymphocytes deficient animals (e.g. CBA/N mice); NK cell deficient animal (e.g. Beige mice); combined immuno-deficient animal (eg. severe combined immuno-deficient (SCID) mice (combined T and B lymphocytes deficient), Beige/Nude (combined T lymphocytes and NK cells deficient), SCID (Severe Combined Immune Deficiency, also known as Prkdcscid) Beige/NOD (Non-Obese Diabetes) SCID mice (combined T, B lymphocytes and NK cells deficient)), and animals which are treated or manipulated to have an immune system which resembles that in any of the above-mentioned immuno-deficient animals. In certain embodiments, the immuno-deficient animals are NOD SCID mice further depleted of Interleukin 2 receptor gamma chain (i.e., NSG (NOD-SCID-Gamma) mice), which are first developed in the laboratory of Dr. Leonard Shultz at The Jackson Laboratory (Shultz L D; Lyons B L; Burzenski L M et al., 2005, “Human lymphoid and myeloid cell development in NOD/LtSz-scid IL2R gamma null mice engrafted with mobilized human hemopoietic stem cells”. J. Immunol. 174 (10): 6477-89; Shultz L D; Schweitzer P A; Christianson S W et al., 1995, “Multiple defects in innate and adaptive immunologic function in NOD/LtSz-scid mice”. J. Immunol. 154 (1): 180-91). NSG mice are deficient in multiple cytokine signaling pathways and hence deficient in innate immunity, which permit the engraftment of a wide range of primary human cells, and enable sophisticated modeling of many areas of human biology and disease in such type of animal model. Examples of different strains of NSG mice include, for example: original NSG mice (developed by The Jackson Laboratory), NPG mice (NOD-Prkdc^{scid} Il2rg^{null} mice developed by Beijing Vitalstar Biotechnology), NOG mice (NOD/Shi-scid/IL-2Rγ^{null} mice developed by Central Institute for Experimental Animals (CIEA)), NCG (NOD-Prkdc^{em26cas9d52}Il2rg^{em26cas9d22}Nju mice developed by Model Animal Research Center of Nanjing University). Different strains of NSG mice are by so far most highly immuno-deficient mice available, they have longer life span than NOD SCID mice which enables them for long term observations, they have basically no rejection to human derived cells or tissue, and in cancer research only a few cancerous cells need to be grafted to NSG mice in order to develop a cancerous lesion, also they are claimed to have no “leaky” lymphocytes and are with extremely low amount of active NK cells. In certain embodiments, the immuno-deficient animals used in herein are NPG mice. In certain embodiments, the immuno-deficient animals used in herein are NCG mice.

The animals provided herein simulate or mimic a human disease treatable through immunomodulation. In certain embodiments, the human disease is a proliferative disease which involves uncontrolled cell growth. In certain embodiments, the human disease is tumor or cancer.

As used herein, a human disease “treatable” through immunomodulation means that immunomodulation can reduce one or more symptoms of the disease, slow down or stop the disease progression, delay the disease onset, reduce the disease lesion, release protective cytokines or antibodies in sufficient amounts to provide protection, and/or induce activation of immune cells against the unwanted disease lesion.

“Immunomodulation” as used herein includes modulation of the immune system, for example, activation or suppression of one or more immune responses. In certain embodiments, immunomodulation involves modulating one or more therapeutic targets involved in one or more immune response pathways. Such therapeutic targets may include, for example, PD-L1 (programmed death-1 ligand 1) and its receptor PD-1, or PD-L2 (Programmed death-1 ligand 2) and its receptor PD-1; TGF-β (Transforming growth factor-beta) and its receptor TGF-β R; RANK (Receptor activator of nuclear factor-κB) and its ligand RANKL; CTLA-4 and its ligand CD80, CD86; CD28 and its ligand CD80, CD86; Lag-3 and its ligand MHC class I or II peptide; GITR and its ligand GITRL; ICOS and its ligand B7RP1; BTLA and its ligand HVEM; CD137 and its ligand CD137L; KIR and its ligand MHC class I or II peptide; DX40 and its ligand OX40L; CD27 and its ligand CD70, TIM3 and its ligand GAL9 and etc. In certain embodiments, immunomodulation involves modulating one or more tumor targets involved in Antibody Dependent Cell Mediated Cytotoxicity (ADCC) effect, for example, including but not limited to antibodies targeting Her2, EGFR, CD38, CD20, CD52 and etc.

The animals provided herein comprise functional human immune cells. The term “immune cells” as used herein is intended to encompass cells related to innate immunity such as NK (natural killer) cells, mast cells, phagocytes (including macrophases, neutrophils, dendritic cells), basophils, eosinophils, and gamma/delta T cells, and cells related to adaptive immunity such as B lymphocytes and T lymphocytes. In certain embodiments, the human immune cells are human PBMC (peripheral blood mononuclear cells) or immunocytes derived from human PBMC. The term “PBMC” as used herein refers to any blood cell having a round nucleus, for example, lymphocytes (typically consists of T cells, B cells and NK cells) and monocytes. These lymphocytes and monocytes recirculate between tissues and blood, and are a critical component in the immune system to fight infection and adapt to intruders. PBMCs may be obtained from whole blood samples by any methods known in the art. For example, PBMCs may be extracted from whole blood using a ficoll gradient or percoll gradient method. In some embodiments, the human immune cells are immunocytes derived from human PBMC (e.g. T lymphocytes, B lymphocytes or NK cells). Subgroups of human immune cells (e.g. T cells or NK cells) can be further separated from human PBMC through any methods known in the art, include but are not limited to, density gradient centrifugation, immunomagnetic separation or fluorescence activated cell sorting. In certain embodiments, the human immune cells are or are derived from activated T cells. For example, T cells can be isolated by incubate PBMC with CD3-antibody for a period of time and then subject all cells to FACS cell sorting program such that to obtain CD3+ T cells. In some embodiments, CD4+ and CD8+ T cells are separated through CD4+ negative selection and CD8+ negative selection respectively. In certain embodiments, CD4+ and CD8+ T cells are cultured and activated separately. In certain embodiments, CD4+ and CD8+ T cells are cultured and activated together. T cells can be activated by suitable methods, include but are not limited to, co-culturing with CD3/CD28 labeled beads, and/or with pro-inflammatory factors (e.g. interleukins, such as IL2, IL6, IL12, IL15, IL21 etc.), and/or with attenuated antigen presenting feeder cells (e.g. irradiated tumor cells, or mitomycin C treated tumor cells).

The term “functional” with reference to immune cells means, the human immune cells are capable of mounting a human humoral immune response and/or cellular immune response to an intended antigen.

In certain embodiments, the human immune cells are derived from one human donor. The human donor is preferably a healthy donor and does not have the human disease for which the animal model is intended to mimic.

The animals provided herein further comprise a human xenograft comprising a pathogenic human cell or tissue.

The term “xenograft” as used herein refers to tissue or cells taken from or derived from a donor which is a species different from the animal model, and are suitable for being grafted into the animal model. In some embodiments, the donor of the xenograft is human. In some embodiments, the human xenograft is derived from a human patient having the disease.

In some embodiments, the xenograft tissue or cells are tumor tissue or cells, or cancerous tissue or cells. The xenograft can be derived from a cell line such as a cancer cell line, or can be derived from a biopsy sample (e.g. a tumor sample) taken from a human patient.

The human immune cells and the human xenograft are selected to meet at least one of the following criteria: i) the human xenograft expresses a threshold level of a therapeutic target for the human disease; ii) the human immune cells match with the human xenograft for at least one human leukocyte antigen (HLA) marker; and iii) the human xenograft cells expresses a desired level of the matched HLA marker.

The therapeutic target can be any biological molecule that participates in the immunomodulation of the human disease. The therapeutic target can be a tumor surface antigen (e.g. Her2, EGFR, CD38, CD20, CD52 and etc) which is involved in Antibody Dependent Cell Mediated Cytotoxicity (ADCC) effect, or proteins involved in immune response pathways (e.g. PD-L1 (programmed death-1 ligand 1) and its receptor PD-1, or PD-L2 (Programmed death-1 ligand 2) and its receptor PD-1; TGF-β (Transforming growth factor-beta) and its receptor TGF-β R; RANK (Receptor activator of nuclear factor-κB) and its ligand RANKL; CTLA-4 and its ligand CD80, CD86; CD28 and its ligand CD80, CD86; Lag-3 and its ligand MHC class I or II peptide; GITR and its ligand GITRL; ICOS and its ligand B7RP1; BTLA and its ligand HVEM; CD137 and its ligand CD137L; KIR and its ligand MHC class I or II peptide; DX40 and its ligand OX40L; CD27 and its ligand CD70, TIM3 and its ligand GAL9 and etc.

The expression of the therapeutic target for the human disease in the human xenograft can be analyzed using suitable methods. For example, when the xenograft is derived from an existing cell line, then the gene expression profile of the cell line can be obtained from publicly available database (for example, XenoBase) and the expression of the interested therapeutic target in the cell line can be hence determined. Alternatively or additionally, the expression of the therapeutic target in the cell line can also be detected at RNA level by methods such as reverse transcription PCR, probe hybridization or other suitable methods known in the art, and/or at protein level by methods such as ELISA, FACS and the like.

In certain embodiments, the human xenograft expresses a threshold level of the therapeutic target. The threshold level can be RNA level and/or protein level. The threshold level can be determined by people skilled in the art based on the purpose of the animal model. For example, if the animal model is for the purpose of mimicking a human disease, then the threshold can be determined to be similar to the expression level of the therapeutic target in the disease setting. For another example, if the animal model is for the purposes of evaluating activity of a therapeutic agent and hence high sensitivity would be desired, then the threshold can be selected to be higher than physiological expression level or even higher than the modest pathological expression level to allow desired sensitivity in the testing.

In certain embodiments, the human immune cells are selected such that they match with the human xenograft for at least one human leukocyte antigen (HLA) marker. “HLA markers” described herein contains different classes, subtypes and alleles of antigens essential for immune function in humans, which control immune responses through antigen presentation and distinguish self from non-self. The HLA marker include for example, HLA class I antigens (subtypes: HLA-A, HLA-B, HLA-C and the alleles thereof (e.g. HLA-A1, HLA-A2, HLA-B27, HLA-B53 and such like)); HLA class II antigens (subtypes: HLA-DP, HLA-DM, HLA-DOA, HLA-DOB, HLA-DQ, HLA-DR and the alleles thereof (e.g. HLA-DRB1, HLA-DQA1, HLA-DQB1, HLA-DPA1, HLA-DPB1 and such like)); and HLA class III antigens (e.g. HLA-C2, HLA-C4 and HLA-Bf) (for more details, please refer to: Complete sequence and gene map of a human major histocompatibility complex: The MHC sequencing consortium (1999) Nature 401, 921-923; Trowsdale, J. (1996) Molecular genetics of HLA class I and class II regions. In: Browning, M. J. and Mcmichael, A. J. eds., HLA and MHC genes, molecules and function. Oxford: Bios Scientific Publishers Ltd., 23-39; Hall, F. C. and Bowness, P. (1996) HLA and diseases: from molecular function to disease association? In: Browning, M. J. and Mcmichael, A. J. eds., HLA, and MHC genes, molecules and function. Oxford: Bios Scientific Publishers Ltd. 353-381, all of which are incorporated herein to entirety). The term “match” with respect to HLA marker, means that the human immune cells share at least one identical HLA marker with the human xenograft. This matching step is optionally performed to ensure the immunological compatibility between the human immune cells and the human xenograft. The matching step can generally be conducted through any methods known in the art, include but are not limited to mRNA expression profiling (e.g. qRT-PCR, probe hybridization and such like) or protein expression detection (e.g. ELISA, FACS and such like) of the HLA markers. For example, the HLA markers can be typed in the immune cells and the xenograft respectively by sequencing the HLA alleles, conducting serological HLA testing and/or DNA genotyping.

In certain embodiments, the matched HLA marker between the human immune cells and the human xenograft is HLA-A2 and/or HLA-B.

In certain embodiments, the human xenograft cells express a desired level of the matched HLA marker. The expression level of the matched HLA marker can be determined at RNA level and/or protein level. In certain embodiments, the matched HLA marker is expressed in the human xenograft at a relatively high level as compared with other candidate xenografts.

In certain embodiments, the human immune cells and the human xenograft meet all the three criteria as described above.

The animal model described above can be used to test or select candidate immunomodulator for efficacy on disease development and progression, or to test the efficacy of an immunomodulator for a disease. In some embodiments, the test or selection are carried out by observing physical changes in the human xenograft in the animal model, and in some other embodiments, the test or selection are carried out by detecting specific immune response resulted from the immunomodulator against the xenograft in the animal.

Methods of Producing the Animal Models

Another aspect of the present disclosure relates to methods of producing an animal model for a human disease. In certain embodiments, the method comprises: a) obtaining an immuno-deficient animal; and b) grafting the animal with functional human immune cells and a human xenograft comprising a pathological human cell or tissue; wherein the human immune cells and the human xenograft meet at lease one of the following criteria: i) the human xenograft expresses a threshold level of a therapeutic target for the human disease; ii) the human immune cells match with the human xenograft for at least one human leukocyte antigen (HLA) marker; and iii) the human xenograft cells expresses a desired level of the matched HLA marker.

The immuno-deficient animal can be obtained either through genetic modification, for example, by introducing a DNA sequence into the animal such that to promote a deficiency in functionally active B/T lymphocytes or/and NK cells. Examples of genetically modified immuno-deficient animals are commercially available BALB/c nude mice, C57BL nude mice, NIH nude mice, nude rat, CBA/N mice, Beige mice, SCID mice, Beige/Nude, SCID Beige/SCID NOD mice, NOD-Prkdc^{scid} IL2Rγ null mice (e.g. NSG mice, NPG mice, NOG mice, or NCG mice), and animals which are treated or manipulated to have an immune system which resembles that in any the above-mentioned immuno-deficient animals. Alternatively, an immuno-deficient animal can be obtained by biological or chemical immuno-suppress methods, include, without limitation, immuno-suppression by repeated treatment with irradiation, mitomycin C, cyclosporine, anti-Asialo GM1 antibody, or other immuno-suppressive agents or treatments well known in the art.

The human immune cells and the human xenograft can be grafted to the animal using any suitable methods known in the art, for example, by grafting cells subcutaneously, intraperitoneally, or intravenously through injection; or alternatively, by implanting a fraction of tissue through surgery.

In some embodiments, the human immune cells are injected intravenously into the animal. In some embodiments, the human immune cells are injected subcutaneously into the animal. In some embodiments, the human immune cells and the xenograft are mixed and implanted subcutaneously. In some embodiments, the human immune cells are cultured and activated before being grafted to the animal. For example, in some embodiments, the human immune cells are T lymphocytes, and before being grafted to the animal, the T lymphocytes are cultured and activated by any method known in the art, include but are not limited to co-culturing with CD3/CD28 labeled beads, and/or with pro-inflammatory factors (e.g. interleukins, such as IL2, IL6, IL12, IL15, IL21 etc.), and/or with attenuated antigen presenting feeder cells (e.g. irradiated tumor cells).

The human xenograft can be implanted through subcutaneously injection. The site of implantation may be into any subcutaneous site which will permit blood supply to reach the implant, such as the flanks of the host animal. In some embodiments, the xenograft is pre-treated before grafting into the animal model. The term “pre-treated” when refers to tissue, generally relates to any processing methods known in the art to treat a tissue before its engraftment, such as washing, homogenization, re-suspension and/or mixing with a solution (eg. saline, PBS etc.) or a matrix (e.g. collagen). The term “pre-treated” when refers to cells, includes any processing methods known in the art to treat cells before its engraftment, such as culture, sub-culture, activating, treatment with an agent, centrifugation, re-suspension, filtration, and mixing with a solution (e.g. saline, PBS etc.) or a matrix (e.g. collagen).

In an illustrative example, the human xenograft is grafted to the left or the right flank of the animal. In some embodiments, for example, the xenograft is a piece of diseased tissue taken through biopsy from a human patient, and the xenograft is washed in PBS for a few times and then homogenized and resuspended in saline or PBS and optionally mixed a small amount of matrigel before being injected under the skin of the animal's upper back or left/right flanks. In other embodiments, the xenograft are tumor cells or cancerous cells, which are cultured and collected before use, the cells are then resuspended in saline or PBS and optionally mixed a small amount of matrigel before being injected under the skin of the animal's upper back or left/right flanks or being injected intravenously.

In certain embodiments, the human immune cells are grafted through single delivery before the human xenograft is grafted, or are grafted through single delivery after the human xenograft is grafted, or are grafted through single delivery simultaneously when the human xenograft is grafted, or are grafted through multiple deliveries before, after or simultaneously when the human xenograft is grafted.

Once grafted with xenografts, the animal model is allowed to grow for a time sufficient to simulate the lesion of the human disease. For example, given enough time for disease development, animal models grafted with cancerous cells may develop in-situ or metastatic tumors which can then be used to evaluate the efficacy of a test agent on the disease. In some embodiments, blood or tissue (especially spleen tissue) are collected from the animals grafted with human immune cells during the assessment of an immunodulatory agent in order to prove the presence of a human immune system in the animal model. For example, blood or/and spleen tissues can be collected and processed to evaluate the proportion of CD4+ cells (helper T cells) and CD8+ cells (cytotoxic T cells) in the whole cell population.

Methods of Using the Animal Models

Another aspect of the present disclosure relates to methods of using the animal models provided herein.

In certain embodiments, the present disclosure provides methods of assessing effect of an immunomodulatory agent on a human disease, comprising: a) obtaining an animal model provided herein for the human disease; b) administering the immunomodulatory agent to the animal model; and c) determining the effect of the immunomodulatory agent on the human xenograft and/or on the animal model.

The term “immunomodulatory agent” as used herein refers to any substance, molecule, element, compound, or a combination thereof capable of suppressing or activating human immune response. The term “immunomodulatory agent” is intended to include both known therapeutic agents and potential therapeutic agents. A test agent can be in any form including, but not limited to, protein, polypeptide, polynucleotide, small organic/inorganic molecule and the like. An immunomodulatory agent can be a natural product, extracts of a natural product, a synthetic compound or a combination of two or more substances, and may include antisense compounds, or antibodies. In some embodiments, the immunomodulatory agent has anti-cancer activity.

The term “anti-cancer activity” refers to activity or effect to inhibit, prevent or suppress cancer cell growth, proliferation or metastasis.

In certain embodiments, the immunomodulatory agent is administered to the animal in combination with a second test agent. The second test agent may or may not be an immunomodulatory agent, and may or may not have anti-cancer activity. The second test agent can be administered before, after, or simultaneously with the immunomodulatory agent to the animal, so long as the effects of both agents overlap in the animal for a sufficient period of time. This would allow assessment of an interested combination of therapeutics in the animal model, and also provide a way to select the proper agent to be combined with an interested immunomodulatory agent.

The method of assessing the effect of an immunomodulatory agent on a human disease comprises determining the effect on the xenograft itself. For example, when the human disease is cancer, the immunomodulatory agent is an active agent for cancer therapy, the effect of the agent is evaluated by assessing the size and/or weight of the human xenografts, and/or assessing the morbidity and mortality of the animal. For example, at the time of routine monitoring, the physical conditions of the animals will be checked for any effects of tumor growth and any changes in normal behavior such as mobility, food and water consumption, body weight gain/loss, eye/hair matting etc. In some embodiments, blood and tissue samples (e.g. the xenograft) are collected for more in-depth assessment of the animal's health condition, parameters to be assessed may include but not limited to, immune system activity, tumor cells biomarkers, pro-inflammatory/anti-inflammatory factors, drug metabolisms, immune cells infiltration, malignancy level, metastasis condition and the like.

The method of assessing the effect of an immunomodulatory agent on a human disease may further comprise determining the effect of the agent on the human immune response against the human xenograft, by detecting expression of a cytokine of interest from the human immune cell, and/or detecting the population of the human immune cells in the animal model, and/or detecting infiltration of the human immune cell to the human xenograft. For example, when the human disease is cancer, the immunomodulatory agent is an active agent for cancer therapy, the effect of the immunomodulatory agent on human immune response can be evaluated by detecting the proportion of CD4+/CD3+ cells (helper T cells), CD8+/CD3+ cells (cytotoxic T cells), CD4+/CD25+/FoxP3+ cells (regulatory T cells), or CD11b+/Ly6G+ myeloid-dewed, suppressor cells (MDSC) in tumor infiltrating lymphocyte, tumor draining lymphnodes (TDL) the whole cell population of a blood sample or a spleen tissue sample, detecting the expression of a cytokine (eg. (interleukins (eg. IL2, IL-4, IL-6 and IL-10, etc); interferons (eg. IFN-γ, IFN-α); tumor necrosis factors (eg. TNF-α) and such like) from the human immune cell in blood samples collected from the animal, and/or detecting the population of the human immune cells in the animal model, and/or detecting infiltration of the human immune cell to the cancerous tissue.

In certain embodiments, the present disclosure provides methods of identifying an active immunomodulatory agent for a human disease, comprising: a) obtaining the animal model provided herein for the human disease; b) administering a test immunomodulatory agent to the animal model; and c) determining the effect of the test agent on the human xenograft; wherein therapeutic improvement in the human xenograft indicates an active immunomodulatory agent.

The term “therapeutic improvement” as used herein refers to a positive outcome of the treatment on the animal model, wherein the animal, or specifically the human xenograft in the animal, demonstrates a reduction in severity, in size, or in a disease-related biomarker, or an improvement in conditions associated with the disease or disorder. In some embodiment, therapeutic improvement refers to an inhibition of tumor growth, progression or metastasis in the animal. In some embodiment, therapeutic improvement refers to a lower morbidity and/or mortality, and/or a longer survival time of the animal.

As used herein, the term “subject” or “individual” or “patient” used interchangeably here refers to a human suffered from a disease, disorder or conditions related to a disease or disorder. In some embodiments, a subject refers to a human suffers from cancer. In some embodiments, the term refers to a human in need of treatment for cancer with an anti-cancer agent.

Unless defined otherwise, all technical and scientific terms used herein have the same meaning as commonly understood by one of ordinary skill in the art to which this invention belongs.

As used herein, the singular forms “a”, “an” and “the” are intended to include the plural forms as well, unless the context clearly indicates otherwise. It will be further understood that the terms “comprises”, “comprising”, “includes” and/or “including,” if used herein, specify the presence of stated features, steps, operations, elements and/or components, but do not preclude the presence or addition of one or more other features, steps, operations, elements, components and/or groups thereof. The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “3000 mm²” is intended to mean “about 3000 mm²”. As used herein, the term “about,” when used in reference to a particular recited numerical value, means that the value may vary from the recited value by no more than 1%.

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

Although any methods and materials similar or equivalent to those described herein can be used in the practice or testing of the present disclosure, preferred methods and materials are now described.

The following examples are presented to illustrate the present disclosure. They are not intended to limiting in any manner. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Identify Candidate Xenograft (Cell Lines to Build Mixeno Models for In Vivo Efficacy Evaluation of Anti-PD-1/Anti-PD-L1 Antibodies

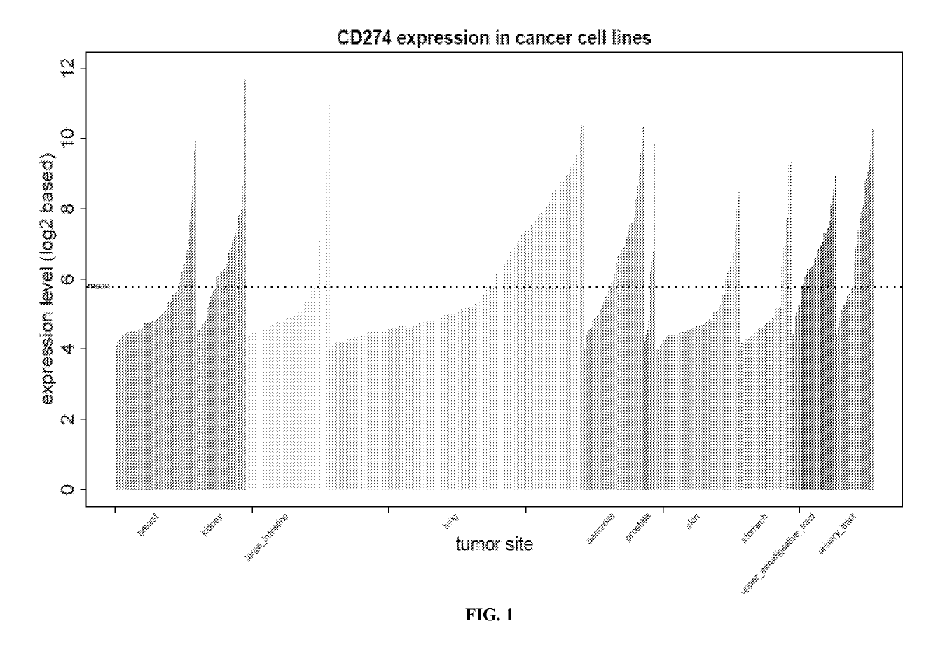

XenoBase is a free web-based tool developed by CrownBio, combining the publically available profiling data of more than 1000 cell lines, with CrownBio proprietary in vivo pharmacology data. In order to select xenografts (in this embodiment, cell lines) meeting the criteria of expressing a threshold level of a therapeutic target (herein PD-L1) for the human disease, 553 cell lines originated from breast, skin, lung, kidney, large intestine, prostate, pancreas, stomach, upper aerodigestive tract and urinary tract were screened for PD-L1 (CD274) mRNA expression level (FIG. 1) for in vivo efficacy evaluation of anti-PD-1 antibody. FACS analysis was performed to further determine the surface PD-L1 protein expression level of 14 cell lines with relatively high PD-L1 mRNA expression, and the results are listed in Table 1.

TABLE 1
PD-L1 mRNA and protein expression level of 14 cell lines

| | | | | PD-L1 protein |
|---|---|---|---|---|
| | | | PD-L1 mRNA | level (Mean |
| | | | Level | Flurescence |
| No. | Cell line | Cancer type | (Xenobase) | Intensity) |
| 1 | SK-MEL-28 | Melanoma | 5.5561 | 7 |
| 2 | A2058 | Melanoma | 5.0168 | 35.9 |
| 3 | A375 | Melanoma | 4.8704 | 13.4 |
| 4 | NCI-H292 | NSCLC | 9.2971 | 136.8 |
| 5 | HCC827 | NSCLC | 8.4718 | 95 |
| 6 | NCI-H358 | NSCLC | 7.5277 | 150.9 |
| 7 | SK-MES-1 | NSCLC | 7.3042 | 157.8 |
| 8 | NCI-H1975 | NSCLC | 6.8753 | 134.8 |
| 9 | NCI-H2228 | NSCLC | 6.3227 | 131.2 |
| 10 | NCI-H1650 | NSCLC | 5.8356 | 31.4 |
| 11 | MDA-MB-231 | TNBC | 8.1606 | 283.1 |
| 12 | 786-O | Kidney Cancer | 7.2959 | 43.7 |
| 13 | A498 | Kidney Cancer | 5.1619 | 19.5 |
| 14 | SCC-4 | H&N | 7.3154 | 26.7 |

As shown in Table 1, the NSCLC cell line HCC827 has relatively high PD-L1 expression in both mRNA and protein level, while the Melanoma cells line A375 has relatively low PD-L1 expression in both mRNA and protein level.

HCC827 cell line with high PD-L1 expression and A375 cell line with low PD-L1 expression were used to generate Mixeno models in Example 2 and 3 respectively.

### Example 2

### Efficacy Evaluation of Anti-PD-1 Antibody in the HCC827 Mixeno NOD/SCID Mouse Model

Cell Culture:

The HCC827 cell line was obtained from American Type culture Collection (ATCC), and maintained in vitro as a monolayer culture in RPMI 1640 medium supplemented with 10% heat inactivated fetal bovine serum, at 37° C. in an atmosphere of 5% CO₂ in air. The tumor cells were routinely subcultured every 3-5 days by trypsin-EDTA treatment. Cells growing in an exponential growth phase were harvested and counted for tumor grafting.

Animals:

NOD-SCID mice purchased from Shanghai Laboratory Animal Center are used for the study, which are all Females, 6-8 weeks old, weighing approximately 15-18 g. A total number of 10 mice are sublethally irradiated with ⁶⁰Co (200 rad) 2 days before tumor cell grafting (day −2) and randomized by weight into 3 groups (Group 1: mice engrafted with human PBMC and isotype control IgG4, n=4; Group 2: mice engrafted with human PBMC and anti-PD-1 antibody, n=3; Group 3: mice engrafted with anti-PD-1 antibody but without being engrafted with human PBMC, n=3).

Human Immune Cells Grafting:

At day −1 (one day before tumor cell grafting), day 12 (12 days after tumor cell grafting) and day 27, 4×10⁶ human PBMC from selected donor were transferred through intravenous injection to Group 1 and Group 2 mice.

Tumor Grafting:

At day 0, each mouse is grafted subcutaneously at the right flank with 1×10⁷ of HCC827 cells in 0.1 ml of PBS.

Immunomodulatory Agent Treatment:

Mice with xenografts were grown 6 days to allow the xenograft to develop in the mice, and then were treated with isotype control (human IgG4) or anti-PD-1 antibody (produced based on the published sequences of Nivolumab: BMS-936558) at 150 ug/mouse twice weekly through intravenous injection starting at day 6, for a total of 9 doses.

Endpoints:

Tumors were measured twice weekly in two dimensions using a digital caliper, starting from day 6, and the volumes were expressed in mm³ using the formula: V=0.5 a×b², where a and b are the long and short diameters of the tumor, respectively. The major endpoint of the study was tumor growth inhibition (TGI). TGI is expressed as: TGI (%)=100×(1−T/C). T and C are the mean tumor volume of the treated and control groups, respectively, on day 34.

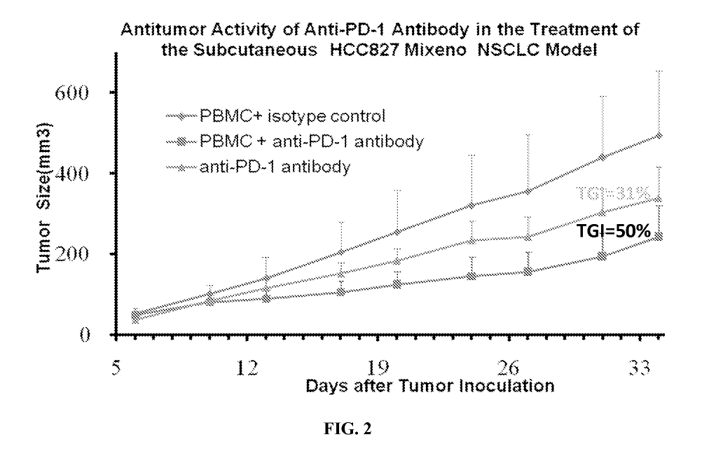

As shown in FIG. 2, among the mice engrafted with human PBMC, the treatment with anti-PD-1 antibody (Group 2) demonstrated 50% tumor growth inhibition (TGI), compared with isotype control treated group (Group 1). The mice not grafted with PBMC but treated with anti-PD-1 antibody (Group 3) showed a TGI of 31%.

This study indicates in the xenograft (HCC827 cell line with high PD-L1 expression) mouse model where immune-deficient mice (NOD/SCID mouse) were engrafted with human PBMC, anti-human PD-1 antibody demonstrated encouraging efficacy (higher than those without PBMC), thus this animal model can be used for in vivo evaluation of immunotherapeutic agents.

### Example 3

### Efficacy Evaluation of Anti-PD-1 Antibody in the A375 Mixeno NOD/SCID Mouse Model

Cell Culture:

The A375 cell line was obtained from Shanghai Institutes for Biological Sciences (SIBS), and maintained in vitro as a monolayer culture in DMEM medium supplemented with 10% heat inactivated fetal bovine serum, at 37° C. in an atmosphere of 5% CO₂ in air. The tumor cells were routinely subcultured every 3-5 days by trypsin-EDTA treatment. Cells growing in an exponential growth phase were harvested and counted for tumor grafting.

Animals:

NOD/SCID mice purchased from Beijing HFK Bioscience are used for the study, which are all females, 8 weeks old, weighing approximately 18-22 g. A total number of 20 mice are sublethally irradiated with ⁶⁰Co (200 rad) 1 day before tumor cell grafting (day −1) and randomized by weight into 4 groups.

Human Immune Cells and Tumor Grafting:

Human PBMCs were isolated from a HLA-A2 positive healthy donor, and T cells were separated from each PBMC samples by CD4+ negative selection and CD8+ negative selection. HLA-A2 positive A375 cells (in this case, both the T cells and the A375 cells were HLA-A2 positive as confirmed by FACS analysis) The MFI of HLA-A2 in A375 cells is 103 (versus the MFI value less or around 10 for those unmatched cell lines; the MFI of HLA-A2 in donor PBMC is 1412 (versus the 2 digit MFI value for those unmatched donor PBMC. In this case, the PD-L1 expression in A375 cell line is not high though (MFI=13.4), but we selected this line because anti-PD-1 antibody (e.g. Nivolumab and Pembrolizumab) has been approved in Japan, USA and UK for the treatment of metastatic or advanced Melanoma, and most of the melanoma cell lines don't have high level of PD-L1 expression compared to many NSCLC cell lines and other cell lines (FIG. 1). T cells were pre-activated through a series of subculture steps including: 1) culturing in RPMI 1640 supplemented with 10% heat inactivated fetal bovine serum and hIL-2; 2) co-culturing with mitomycin C treated A375; 3) co-culturing with anti-CD3/28 beads and in presence of IL-2; and repeat steps in order of 2) to 3) and then to 1). In Groups 3 and 4, 5×10⁶ of pre-activated T cells were mixed with 5×10⁶ of A375 cells in 0.1 ml of PBS and grafted subcutaneously at the right flank of the mice. In Group 1 and 2, 5×10⁶ of A375 cells (without human immune cells) in 0.1 ml of PBS were grafted subcutaneously at the right flank of the mice.

Immunomodulatory Agent Treatment:

Mice of Groups 1 and 3 were treated with isotype control (human IgG4) and mice of Groups 2 and 4 were treated with anti-PD-1 antibody (produced based on the published sequences of MPDL3280A) at 1 mg/kg twice weekly through intravenous injection starting at day 0, day 3, day 5, day 8, day 10, day 12 and day 15 for a total of 7 doses.

Endpoints:

Tumors were measured twice weekly in two dimensions using a digital caliper, starting from day 7, and the volumes were expressed in mm³ using the formula: V=0.5 a×b², where a and b are the long and short diameters of the tumor, respectively. The major endpoint of the study was tumor growth inhibition (TGI). TGI is expressed as: TGI (%)=100×(1−T/C). T and C are the mean tumor volume of the treated and control groups, respectively, on day 21.

TABLE 2
TGI and statistical analysis in the absence
of T cell engraftment (NOD/SCID)

| | Tumor Size (mm3) | | |
|---|---|---|---|
| Treatment | at day 21 | TGI (%) | P value |
| No T Cell + isotype | 816 ± 90 | — | — |
| (Group 1) | | | |
| No T Cell + PD-L1 Ab | 867 ± 112 | −6 | 0.731 |
| (Group 2) | | | |

TABLE 3
TGI and statistical analysis in the presence
of T cell engraftment (NOD/SCID)

| | Tumor Size (mm³) | | |
|---|---|---|---|
| Treatment | at day 21 | TGI (%) | P value |
| T Cell (s.c.) + isotype | 778 ± 83 | — | — |
| (Group 3) | | | |
| T Cell (s.c.) + PD-L1 Ab | 634 ± 46 | 18 | 0.169 |
| (Group 4) | | | |

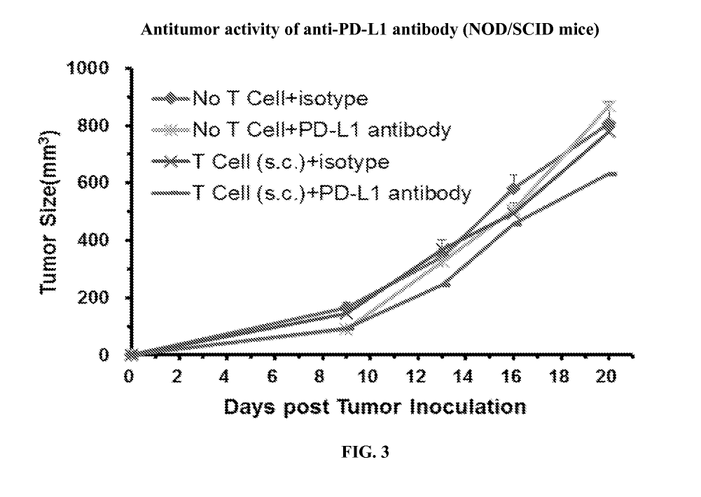

As shown in FIG. 3 and Table 2, in the absence of T cell transfer, the treatment with anti-PD-L1antibody (Group 2) didn't produce any antitumor activity compared with its isotype control treated group (Group 1). And, as shown in FIG. 3 and Table 3, with the engraftment of activated T cells, the treatment with anti-PD-L1 antibody (Group 4) demonstrated slightly tumor growth inhibition with no statistically significance (TGI=18%, p=0.169), compared with the isotype control treated group (Group 3).

By comparing the result obtained in Example 3 with the result in Example 2, it seems that to evaluate an immunomodulatory agent in a Mixeno NOD-SCID mice model, it is preferable to use xenografts expressing a higher level of therapeutic target (herein PD-L1) for the immunomodulatory agent.

### Example 4

### Efficacy Evaluation of Anti-PD-1 Antibody in the A375 Mixeno NPG Mouse Model

Cell Culture:

The A375 cell line was obtained from Shanghai Institutes for Biological Sciences (SIBS), and maintained in vitro as a monolayer culture in DMEM medium supplemented with 10% heat inactivated fetal bovine serum, at 37° C. in an atmosphere of 5% CO₂ in air. The tumor cells were routinely subcultured every 3-5 days by trypsin-EDTA treatment. Cells growing in an exponential growth phase were harvested and counted for tumor grafting.

Animals:

NPG mice purchased from Beijing Vitalstar Biotechnology are used for the study, which are all males, 6-7 weeks old, weighing approximately 19-31 g. A total number of 20 mice) were randomized by weight into 4 groups.

Human Immune Cells and Tumor Grafting:

Human PBMCs were isolated from healthy donors, and T cells were separated from each PBMC samples by CD4+ negative selection and CD8+ negative selection. In this case, both the T cells and the A375 cells were HLA-A2 positive as confirmed by FACS analysis). The MFI of HLA-A2 in A375 cells is 103 (versus the MFI value less or around 10 for those unmatched cell lines; the MFI of HLA-A2 in donor PBMC is 1412 (versus the 2 digit MFI value for those unmatched donor PBMC. In this case, the PD-L1 expression in A375 cell line is not high though (MFI=13.4), but we selected this line because anti-PD-1 antibody (e.g. Nivolumab and Pembrolizumab) has been approved in Japan, USA and UK for the treatment of metastatic or advanced Melanoma, and most of the melanoma cell lines don't have high level of PD-L1 expression compared to many NSCLC cell lines and other tumor cell lines (FIG. 1). T cells were pre-activated through a series of subculture steps including: 1) culturing in RPMI 1640 supplemented with 10% heat inactivated fetal bovine serum and hIL-2; 2) co-culturing with mitomycin C treated A375; 3) co-culturing with anti-CD3/28 beads and in presence of IL-2; and repeat steps in order of 2) to 3) and then to 1). In Groups 3 and 4, 5×10⁶ of pre-activated T cells were mixed with 5×10⁶ of A375 cells in 0.1 ml of PBS and grafted subcutaneously at the right flank of the mice. In Group 1 and 2, 5×10⁶ of A375 cells (without human immune cells) in 0.1 ml of PBS were grafted subcutaneously at the right flank of the mice.

Immunomodulatory Agent Treatment:

Mice of Groups 1 and 3 were treated with isotype control (human IgG4) and mice of Groups 2 and 4 were treated with anti-PD-1 antibody (produced based on the published sequences of MPDL3280A) at 1 mg/kg twice weekly through intravenous injection starting at day 0, day 3, day 5, day 8, day 10, day 12 and day 15 for a total of 7 doses.

Endpoints:

Tumors were measured twice weekly in two dimensions using a digital caliper, starting from day 7, and the volumes were expressed in mm³ using the formula: V=0.5 a×b², where a and b are the long and short diameters of the tumor, respectively. The major endpoint of the study was tumor growth inhibition (TGI). TGI is expressed as: TGI (%)=100×(1−T/C). T and C are the mean tumor volume of the treated and control groups, respectively, on day 21.

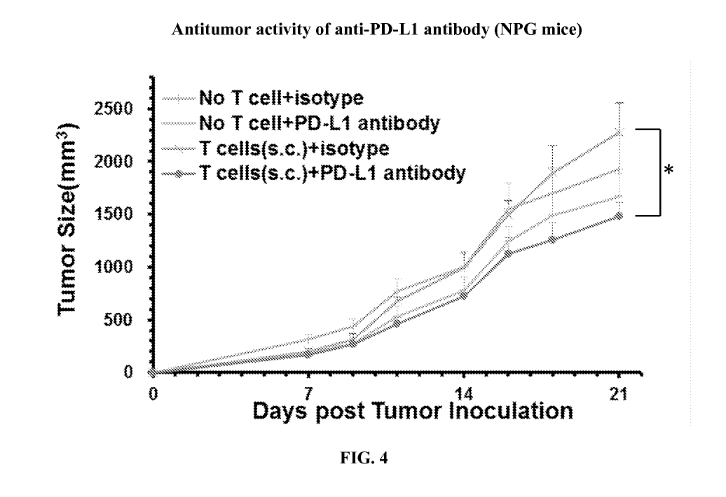

As shown in FIG. 4 and Table 4, in the absence of T cell transfer, the treatment with anti-PD-L1antibody (Group 2) didn't produce much antitumor activity (TGI=14%, p=0.505) compared with its isotype control treated group (Group 1).

TABLE 4
TGI and statistical analysis in the
absence of T cell engraftment (NPG)

| | Tumor Size (mm3) | | |
|---|---|---|---|
| Treatment | at day 21 | TGI (%) | P value |
| No T Cell + isotype | 1932 ± 312 | — | — |
| (Group 1) | | | |
| No T Cell + PD-L1 Ab | 1667 ± 215 | 14 | 0.505 |
| (Group 2) | | | |

However, as shown in FIG. 4 and Table 5, with the engraftment of activated T cells, the treatment with anti-PD-L1 antibody (Group 4) demonstrated significant tumor growth inhibition (TGI=35%, p=0.031), compared with the isotype control treated group (Group 3).

TABLE 5
TGI and statistical analysis in the
presence of T cell engraftment (NPG)

| | Tumor Size (mm³) | | |
|---|---|---|---|
| Treatment | at day 21 | TGI (%) | P value |
| T Cell (s.c.) + isotype | 2281 ± 276 | — | — |
| (Group 3) | | | |
| T Cell (s.c.) + PD-L1 Ab | 1483 ± 131 | 35 | 0.031 |
| (Group 4) | | | |

This study indicates anti-human PD-L1 antibody achieved clear T cell dependent antitumor activity in Mixeno NPG mice model.

By comparing the results obtained in Example 4 with the results in Example 3, it can be seen that using the same anti-PD-L1 antibody with the same dosing regimen didn't achieve significant anti-tumor activity in the A375 MiXeno Model established in NOD/SCID mice as in the A375 MiXeno Model established in NPG mice, demonstrating that NPG mice (or other NOD-Prkdc^{scid} Il2rg^{null} mice) might be a more suitable strain for the efficacy evaluation of immunomodulatory agents.

Result obtained in Example 4 also proved that even if the xenograft are not pre-screened for the higher expressing level of therapeutic target, with MiXeno Model established in NPG mice, one can still expect good quality efficacy data regarding the immunomodulatory agents.

### Example 5

### Adoptive T Cell Transfer (i.v.) and T Cell Reconstitution in NPG (NOD-Prkdc^{scid} Il2rg^{null}) Mice

Animals:

NPG mice purchased from Beijing Vitalstar Biotechnology were used for the study, which are all males, 6-7 weeks old, weighing approximately 24-30 g. A total number of 6 mice were used in this study.

Human Immune Cells Grafting:

Human PBMCs were isolated from healthy donors. T cells were separated from PBMC samples and pre-activated by co-culturing with anti-CD3/28 beads and in presence of IL-2. At days 0 and 1, pre-activated T cells were grafted to the mice through intravenous injection.

Sample Collection:

Blood samples were collected at day 6 (mice #1, 2, 3, 4), day 17 (mice #3, 4, 5, 6) and day 20 (mice #5, 6) for FACS analysis (human CD3 staining) Two mice were sacrificed and spleen were collected per each time point (day 6, 17 and 20) for FACS analysis (human CD3 staining).

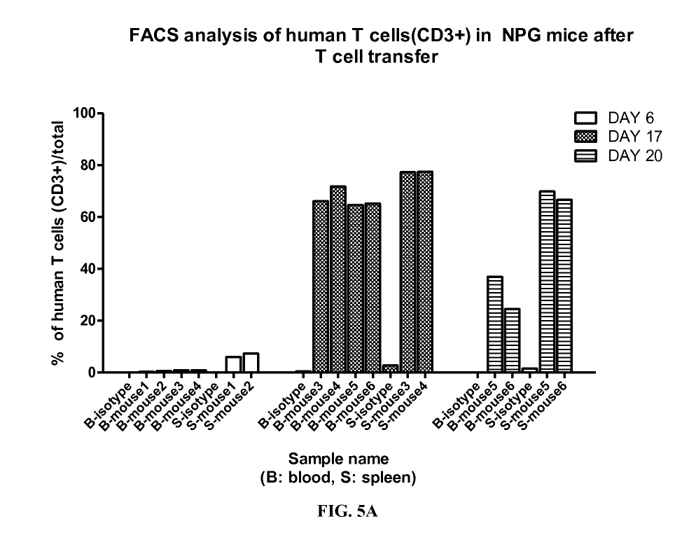

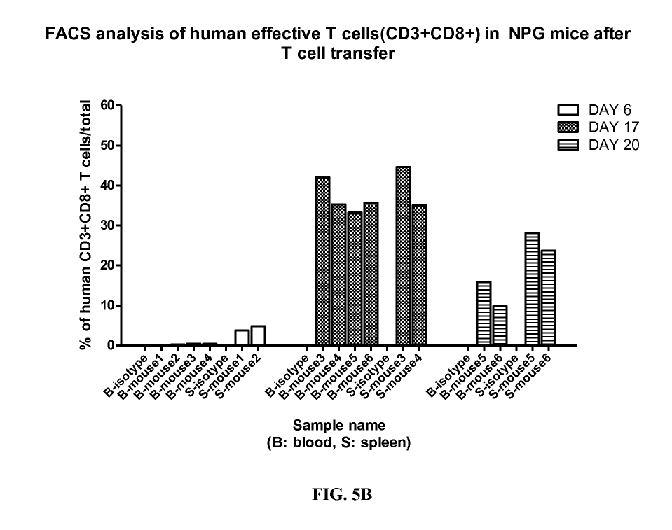

As shown in FIG. 5A, the level of human T cells in peripheral blood peaked at day 17; the percentage of human CD3+ cell population reached >60% of the total counted cells. The reconstitution of human T cells keep maintaining at the level of 30-40% till day 20. The level of human T cells in spleen also peaked at day 17; the percentage of human CD3+ cell population reached around 80% of the total counted cells. The reconstitution of human T cells keep maintaining at the level of >60% till day 20.

As shown in FIG. 5B, at day 17 post T cell injection, among the human T cell population, CD3+/CD8+ human T cells (T_{effect}) reached to 40-50% in both peripheral blood and spleen.

This study indicates that high T cell reconstitution through intravenous injection transfer was well established in the immuno-deficient NPG mice, and the animal models could be developed in T cell transferred (through intravenous injection) NPG mice and used for efficacy evaluation of the immunotherapeutic agents.

While the invention has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. An animal model for a human disease, said animal model comprising an immuno-deficient animal comprising:
(a) functional human immune cells derived from a first human subject; and
(b) a human xenograft derived from a second human subject, said human xenograft comprising a pathogenic human cell or tissue;
wherein:
i) the human xenograft expresses a threshold level of a therapeutic target for the human disease; and/or
ii) the human immune cells match with the human xenograft for at least one human leukocyte antigen (HLA) marker; and/or
iii) the human xenograft cells expresses a desired level of the matched HLA marker.

2. The animal model of claim 1, wherein the human immune cells and the human xenograft meet all three criteria.

3. The animal model of claim 1, wherein the matched HLA marker is HLA-A2 and/or HLA-B.

4. The animal model of claim 1, wherein the human immune cells comprises isolated PBMC or activated T cells.

5. The animal model of claim 1, wherein the matched HLA marker is expressed in the human xenograft at a level higher than in unmatched human xenograft.

6. The animal model of claim 1, wherein the human xenograft comprises a tumor cell or tumor tissue.

7. The animal model of claim 1, wherein the first human subject is different from the second human subject.

8. The animal model of claim 1, wherein the first human subject is the same as the second human subject.

9. The animal model of claim 1, wherein the human immune cells or the human xenograft are grafted to the animal intravenously or subcutaneously or intraperitoneally.

10. The animal model of claim 1, wherein the animal is mouse, a rat, a guinea pig, a hamster, or a dog.

11. The animal model of claim 1, wherein the animal is NOD-Prkdc^{scid} IL2Rγ null mice.

12. The animal model of claim 1, wherein the NOD-Prkdc^{scid} IL2Rγ null mice is selected from a group consisting of strains: NSG (NOD SCID Gamma) mice, NPG (NOD-Prkdc^{scid} Il2rg^{null}) mice, NOG mice (NOD/Shi-scid/IL-2Rγ^{null}), or NCG (NOD-Prkdc^{em26cas9d52}Il2rg^{em26cas9d22}nju) mice.

13. A method of producing an animal model for a human disease comprising:
a) obtaining an immuno-deficient animal; and
b) grafting the immuno-deficient animal with functional human immune cells and a human xenograft comprising a pathological human cell or tissue;
wherein:
i) the human xenograft expresses a threshold level of a therapeutic target for the human disease; and/or
ii) the human immune cells match with the human xenograft for at least one human leukocyte antigen (HLA) marker; and/or
iii) the human xenograft cells expresses a desired level of the matched HLA marker.

14. The method of claim 13, wherein the immuno-deficient animal is depleted of active endogeneous T cells, active endogeneous B cells and active endogeneous Natural Killer cells.

15. The method of claim 13, wherein the human xenograft and the human immune cells the human immune cells and the human xenograft meet all three criteria.

16. The method of claim 13, wherein the matched HLA marker is HLA-A2 and/or HLA-B.

17. The method of claim 13, wherein the human immune cells or the human xenograft are grafted intravenously or subcutaneously or intraperitoneally.

18. The method of claim 13, wherein the human immune cells are grafted through single delivery before, after or simultaneously when the human xenograft is grafted, or are grafted through multiple deliveries before, after or simultaneously when the human xenograft is grafted.

19. The method of claim 11, further comprising allowing the human xenograft to grow for a time sufficient to simulate the lesion of the human disease.

20. A method of assessing effect of an immunomodulatory agent on a human disease, comprising:
a) obtaining an animal model of claim 1 for the human disease;
b) administering the immunomodulatory agent to the animal model; and
c) determining the effect of the immunomodulatory agent on the human xenograft and/or on the animal model.

21. The method of claim 20, further comprising detecting expression of a cytokine of interest from the human immune cell, and/or detecting the population of the human immune cells in the animal model and/or infiltration of the human immune cell to the human xenograft.

22. A method of identifying an active immunomodulatory agent for a human disease, comprising:
(a) obtaining the animal model of claim 1 for the human disease;
(b) administering a test immunomodulatory agent to the animal model; and
(c) determining the effect of the test agent on the human xenograft;
wherein therapeutic improvement in the human xenograft indicates an active immunomodulatory agent.
